SEQUENCE LISTING (5 sequences)

SEQ ID NO: 1 - DNA, 5277 bp, D. melanogaster
  misc_feature (1)...(5277): n = A,T,C or G
aagaacattgatatcgggactttccctaccaccattcaccgccaccagccccgaaaaaccacctgccaccagtctctataactcccgcccccacccccaaacaccatccccccggactagcaaactaactgtattttccccttggtaaaccccctggtaaacccgaaaccgccaagataagccgtctaatcgagaagctgcgccagaccaacaaacggaaaatgttctgctcgctgmthcyssrugcccagacgctcttctgcgtcctgctcatacagggcggcctctatgccaagnthruhcysvauugngygyutyraactccatctgagcggaaacggaaatggcaacgggaacgggaatgggaacuhsusrgyasngyasngyasngyasngyasngyasnggcgagaagtcgctgggcgaagccaaggcagtgcaggtgcccagtcatgyguyssrugyguaaysaavagnvarsrhscccgttggaccctcgacgggagtagccgccgcctggcagaccgcacccrvagyrsrthrgyvaaaaaaatrgnthraartcgcagcagcagcagcaccaacagcatcccggaggatctccgcccggcsrgngngngnhsgngnhsrgygysrrrgyggtggatcctcgccgcatggccatcggtccgccgcagcggccgccttcgygysrsrrhsgyhsargsraaaaaaaaaahgatgccaccaccgtgcggctgaacaaggagatagcggatatggcccagasaathrthrvaarguasnysguaaasmtaagnctggagcgagagcgactaatgctcctggcccgcgccaagcagacggcguguargguargumtuuaaargaaysgnthraagaacagccgccggcgggcagtaatcatcgcggtcatcccttcattgccgugnrraagysrasnhsarggyhsrhaaggccggatacagatgcagcccagcgaggagctggaagtgggcgactatgyarggnmtgnrsrguguuguvagyastyrccggcgctgttgcagcaggccgcacggggctacatcttcggtaatgtgraauugngnaaaaarggytyrhgyasnvacagaagcaaccaccgacgccggttaccctggaacaggattatgagcaggnysgnrrthrrvathrugugnastyrgugnctgagggatctggagaatagacccgttaaatactacggaagagtggtguargasuguasnargrvaystyrtyrgyargvavacagccagcggagagggaggagcaggtggaggctctctacgaaccgctggnraaguarggugugnvaguaautyrgurucactcgttcggcgactttgatgacttctcggaactggagcaggaaccghssrhgyashasashsrguugugngurcaggagccgcacttgggcgaggatgagttgctgcgtgaactggatgccgngurhsugyguasguuuargguuasaatatcaatatcacggcctggaggacgaaacccttccggagaatccctattyrgntyrhsgyuguasguthrurguasnrtyrcgcacgctggagcaattggagctgggtgcacccgactccctgcagcagargthrugugnuguugyaarassrugngnctcttcgagcaggaacaggagcagcgagatgcccccttcaaggcgatguhgugngugngugnargasaarhysaamtcacaccggcaaaatccacaacccgggcaactacaacataagggtgcaghsthrgyyshsasnrgyasntyrasnargvagncaaaagtgggcgaggaaacgaaatggccagggcattgcccataatcctgnystraaargysargasngygngyaahsasnrgaggcggaaaagcagctgcagcgccagatcttcgcccagcacttcaagguaaguysgnugnarggnhaagnhshysccacagcgcaatggcaaactatcgctgtccgccaactcggaggccacgrgnargasngyysusrusraaasnsrguaathrgccagcaagcacctgaaaccatccactcgctccggctccaaggataacaasryshsuysrsrthrargsrgysrysasasngagaagccacatagcgagctaggtagcagtgcatcctcggccgtctccguysrhssrguugysrsraasrsraavasrtccgccacttcttcctccggcaaacaagccgccggcgatggactacctsraathrsrsrsrgyysgnaaaagyasgyuraaacagcagcaggtggagcaacagtcgcagcaggacaaaaaggatcccysgngngnvagugngnsrgngnasysysasragccataagcgatccggttccggatcggcgggatccatgggatcgccasrhsysargsrgysrgysraagysrmtgysrrggaggcggttactcagcgcagccaccgatttcacacagcatagccagcgygygytyrsraagnrrsrhssraasrcagctgatgctgcgcaccgcccgcggtcaacggcaatacgatgtgccagnumtuargthraaarggygnarggntyrasvarcaaatcgagtgtcctaccgccatggatggcatggagcgattcgcctgtgngucysrthraamtasgymtguarghaacysccgattcccgaccggcagggcagataccgctgcatagacgatcatgtgrrasarggngyargtyrargcysasashsvactgtgcgatggcttcatcgactgtcccgatggcgaggacgaggaccgaucysasgyhascysrasgyguasguasargagatcctgcatgttctacaagacgacgaaggcccatctggatgtgttaargsrcysmthtyrysthrthrysaahsuasvaugcggatgcgttactacgctgggcgagagggcgttaagctccaccaaasaauuargtraaarggyargcacccaccaaccacacacacacatacacacacacacaccgtctaatacacagcacacacagagaaaatacacctagcgaactcacattgacaaacaattagttttaacatgtcgccccaagcaaacaagaaccctgttaagttaaacattatcctggaaccagacacacagacaagccacaaaccacccgcctatcctttgatgtccttcatgagaaagattttcccgagcaaataccgtaaaaaaatccccagccacacaaaaattcagctcgcaatgggctaaatggacaattgccaagtatgaaaagcgaaagctatgattaaaaacaaagtgaggcagaaaatgtgcttgacgaaaggtatgattattattacactattattattaaaaaaattaacttaatgaaaagcccaagcagaagaagcagttatacatactaaagctaaattacaattcagcaaaaggacaaaaaaaaaacgatgtttttttcattttcaaacttgtttcgcaggaacagaaaacataatgaggaaaaacaaaattgggcctaactatttttagtatatattaaataaatatattataaatatatcttactatatgaaatatatttcgtgtacattatttaattatacgtgtattaaagttaaataatgttaatgttaaataaacaaatgaaatgaaatgaaatcaaaacgaagcaagcaaaaagaaaaaaagcgataaaaataacgatgaggcagcaagcagaagacgaatgaattatatatacatatacacagaaaaaaaccgacattaaatatatattaatagtatacatataccaataacaaacaaatatacaattgaagaaaatattgcattaggttttattgctgataactgaacaagctaacgagtgcaatataatatacttatatacaatgcaataacagcaagattagcgacaaaaacaaacatttacaacttatgagaatgaagcaaacatttacataaagaaaaagacttgacttcattgaaaacccaattaagtttctatagtactcagatacttttggttttgaacttcaactctcgttgttagtaatacgtttgccattatagatgatttgctccgattctttgtccttcatgtttgtaaataatactttcaattatccttttcgtttgcctgtaaggatctctccccctccataccacagtttttgatttcgttcatgagtgacaacagttctcacggaaatggcagctatctgtagttaagtaaatttatttacataattgaactatgtttaaataacaatggacttataaatatatatatctatatatatatatatatatataaaacgataagcttataagcgaaacgaaaagcttgtacaaatgctaactaacactaatccattctcgaccttttttcgaagccacaagtgtttattcctaatcgatttttgcattagttttttattcgcgctgtttttctcgattggaaaagctgaaactcaaccttaaaactcaacaaacaatgttatctggtccatgaaaattattcgctattattatgcctgttaagtggcgagtttccaacacaatcaatcaattatgaaatacaatcaagcaaatttttaagccgaaatttacaaattaaaatttttaaacggttgtgtacgcaaacaaatgacagcggcaaaagcaaaaagcgcaagataataccaatggcgaattaacaataataatgaataacctctagagcgattttcacatactgcatttcccgcgttttcccccgaatttccgcattaatttccgcatttcattcaattgaaatattgaaacacggcaagcaagcgttttaatcgcgattgactttgtggacttaatcaatcgataattggtgtcatttatttttttgcacgtatgtatttgcatgttactaaatcgattcgtgtgaattgctaaatcctaaaaaaaatgtttaattatttacaacggaataacatatgaaattgcgagcaataaaatttcatatnttatatacagtacgtataaaaaatcgcaaacaaattatatatatattctaattgagcatacgtttacgagtaagtttttatttcacatcgaccccaatctcctgcccttctatatcaacgatcttcgtattaaccgaaaggataaataggggataacctaggtgtttcctttgaaatgattgattaatgattcagtaaaacgtttccataaataactgcaaaataacccacctttaaattaaaaga

SEQ ID NO: 2 - protein, 560 aa, D. melanogaster
MFCSLAQTLFCVLLIQGGLYALHLSGNGNGNGNGNGNGEKSLGEAKAVQVPSHPVGPSTGVAAAWQTAPSQQQQHQQHPGGSPPGGGSSPHGHRSAAAAAFDATTVRLNKEIADMAQLERERLMLLARAKQTAEQPPAGSNHRGHPFIAGRIQMQPSEELEVGDYPALLQQAARGYIFGNVQKQPPTPVTLEQDYEQLRDLENRPVKYYGRVVQPAEREEQVEALYEPLHSFGDFDDFSELEQEPQEPHLGEDELLRELDAYQYHGLEDETLPENPYRTLEQLELGAPDSLQQLFEQEQEQRDAPFKAMHTGKIHNPGNYNIRVQQKWARKRNGQGIAHNPEAEKQLQRQIFAQHFKPQRNGKLSLSANSEATASKHLKPSTRSGSKDNEKPHSELGSSASSAVSSATSSSGKQAAGDGLPKQQQVEQQSQQDKKDPSHKRSGSGSAGSMGSPGGGYSAQPPISHSIASQLMLRTARGQRQYDVPQIECPTAMDGMERFACPIPDRQGRYRCIDDHVLCDGFIDCPDGEDEDRRSCMFYKTTKAHLDVLADALLRWARGR

SEQ ID NO: 3 - DNA, 544 bp, M. musculus
  misc_feature (1)...(544): n = A,T,C or G
agaatgggcctttctgactcattcactcacttcgttttcattatcccaagaaaggcagaaagtgatgtatcctagacactaagaggagagaaattggaggctgctaggaactgaccaagagagaagggtaagggtggctggttcccaagtcagcaggcatcctgtgatggtgtcctgtctgcaggccaaantgtcagccccaggcccttcccacccatgccgtgtggccctgggcaggtgccctgtgatgtgttgggctgtgtggagcaggaanagctgtgtgatgggagggaagantgtcttgatggatccgatgagcagcactgtggtgagctggagagattacatcacacactgggcatgccaaganggtttcaggtctgggaagctcatatgggaaggatataagaggggctggagcagcagggaatggggagacattgttaaaaatgactaggagcaccagagtctttgaagatgatgaagagaccggctaggcatcgggtcccagcctgttttgttaaacctatttgctgcnagggta

SEQ ID NO: 4 - DNA, 141 bp, H. sapiens
gccccaggacccttcccacctgtgcagtgtggccccggccagacgccctgtgaggtgctgggctgcgtggaacaggcgcaggtgtgtgatggcagggaggattgcctcgacggctccgacgagaggcactgcggtgagctg

SEQ ID NO: 5 - protein, 4123 aa, H. sapiens
PLPKPVLKATALPCGSASYRQAQLMPQQEAWRSAAPGPGDKAGGMAAPRPAAAAPADTCQRLTVGVSLPGSASSPALLQPLAGAVGQLWSQHQRPSATCASWSGFHYRTFDGRHYHFLGRCTYLLAGAADSTWAVHLTPGDRCPQPGHCQRVQVTMGPEEVLIQAGNVSVKGQLVPEGQSWLLHGLSLQWLGDWLVLSGGLGVVVRLDRTGSISISVDHELWGQTQGLCGLYNGWPEDDFMEPGGGLAMLAATFGNSWRLPGSEVSPAEYHEACLFAYCAGAMAGSGQEGRQQAVCATFASYVQACARRHIHIRWRKPGFCERLCPGGQLYSDCVSLCPPSCEAVGQGEEESCREECVSGCECPRGLFWNGTLCVPAAHCPCYYCRQRYVPGDTVRQLCNPCVCRDGRWHCAQALCPAECAVGGDGHYLTFDGRSYSFWGGQGCRYSLVQPYPSTCPTPTIRPPVPGAVLVNGQDVGLPWIGAEGLSVRRASSAFLLLRWPGAQVLWGLSDPVAYITLDPRHAHQVQGLCGTFTQNQQDDFLTPAGDVETSIAAFASKFQVAGKGRCPSEDSALLSPCTTHSQRHAFAEAACAILHSSVFQECHRLVDKEPFYLRCLAAVCGCDPGSDCLCPVLSAYARRCAQEGASPPWRNQTLCPVMCPGGQEYRECAPACGQHCGKPEDCGELGSCVAGCNCPLGLLWDPEGQCVPPSLCPCQLGARRYAPGSATMKECNRWELVYAPGACLLTCDSPSANHSCPAGSTDGCVCPPGTVLLDERCVPPDLCPCRHSGQWYLPNATIQEDCNVCVCRGRQWHCTGQRRSGRCQASGAPHYVTFDGLAFTYPGACEYLLVREASGLFTVSAQNLPCGASGLTCTKALAVRLEGTVVHMLRGTRVLVQLSPQFRGRVAGLCGDFDGDASNDLRSRQGVLEPTAELAAHSWRLSPLCPEPGDLPHPCTMNTHRAGWARARCGALLQPLFTLCHAEVPPQQHYEWCLYDACGCDSGGDCECLCSAIATYADECARHGHHVRWRSQELCCLHQTPCALHGGHLGQPAWCGCILLPLCLSDPRLSPLHPALQCEGGQVYEACGPTCPPTCHEQHPEPGWHCQVVACVEGCFCPEGTLLHGRCVLSTCQEGQWHCGGDGGHCEELVPACAEGEALCQENGHCVPHGWLCDNQDDCGDGSDEEGECLCPCVEATGLVSPCTCCAAPGCGEGQMTCSSGHCLPLALLCDRQDDCGDGTDEPSYPCPQGLLACADGRCLPPALLCDGHPDCLDAADEESCLGQVTCVPGEVSCVDGTCLGAIQLCDGVWDCPDGADEGPGHCPLPSLPTPPASTLPGPSPGSLDTASSPLASASPAPPCGPFEFRCGSGECTPRGWRCDQEEDCADGSDERGCGGPCAPHHAPCARGPHCVSPEQLCDGVRQCPDGSDEGPDACVEAPAPPAMRGPPGQAGGPTSSRAPSPPSPPEAQGEGRKGQERSRTHLTVPAGSTQLPLCPGLFPCGVAPGLCLTPEQLCDGIPDCPQGEDELDCGGLPALGGPNRTGLPCPEYTCPNGTCIGFQLVCDGQPDCGRPGQVGPSPEEQGCGAWGPWSPWGPCSRTCGPWGQGRSRRCSPLGLLVLQNCPGPEHQSQACFTAACPVDGEWSTWSPWSVCSEPCRGTMTRQRQCHSPQNGGRTCAALPGGLHSTRQTKPCPQDGCPNATCSGELMFQPCAPCPLTCDDISGQVTCPPDWPCGSPGCWCPEGQVLGSEGWCVWPRQCPCLVDGARYWPGQRIKADCQLCICQDGRPRRCRLNPDCAGEALPSGSLVLSLDRPAAHPPPPSGSDCWPSLSGLWLVLLVTLGQVPGPLWKPEHPVVLPELQQPPPLRPRSPVPWHPPQGTQTEPCEGCEHQGQVHRVGERWHGGPCRVCQCLHNLTAHCSPYCPLGSCPQGWVLVEGTGESCCHCALPGENQTVQPMATPAAAPAPSPQIRFPLATYILPPSGGSCRPLSSPTPACLSLLHPDPCYSPLGLAGLAEGSLHASSQQLEHPTQAALLGAPTQGPSPQGWHAGGDAYAKWHTRPHYLQLDLLQPRNLTGILVPETGSSNAYASSFSLQFSSNGLHWHDYRDLLPGILPLPKVSPAQGRWGQQPTMPFCGFHSLCPQGPSSVPEGHGLHSMLVEYLLFPRNWDDLDPAVWTFGRMVQARFVRVWPHDVHHSDVPLQVELLGCEPGVGLRCASGECVLRGGPCDGVLDCEDGSDEEGCVLLPEGTGRYTVAGRAAHALGLAFEGTAMWEGPGTAFTPKVPRPCMLRSCSRGLAETEHWPPGQESPTSPTEAWDTLSRAPTFLSWEGELGKPHLPLPTPTETRPVSPGPASGVPHHGESVQMVTTTPIPQMEARTLPPGMAAVTVVPPHPVTPATPAGKMPSPSTCVQGLCQSVAPGPFPPVQCGPGQTPCEVLGCVEQAQVCDGREDCLDGSDERHCGELLEGLLSCGALCSPSQLSCGSGECLSAERRCDLRPDCQDGSDEDGCVDCVLAPWSVWSSCSRSCGLGLTFQRQELLRPPLPGGSCPRDRFRSQSCFVQACPVAGAWAMWEAWGPCSVSCGGGHQSRQRSCVDPPPKNGGAPCPGASQERAPCGLQPCSGGTGKGVLGWGHGGSTVGTGRLGLPAPRLTWCPSPTRLRAGPCVCECRSVPEGAGAPMPTLLPGSQGQQKLQWALCGSLPSLLCPLGLSALFHILPGCRCPPGLLLHDTRCLPLSECPCLVGEELKWPGVSFLLGNCSQCVCEKGELLCQPGGCPLPCGWSAWSSWAPCDRSCGSGVRARFRSPSNPPAAWGGAPCEGDRQELQGCHTVCGTGIAGSLGAGVPPSSSQFCTLRTHGMGPTDHSTWGIEVFGWTPWTSWSSCSQSCLAPGGGPGWRSRSRLCPSPGDSSCPGDATQEEPCSPPIECTGFCAPGCTCPPGLFLHNASCLPRSQCPCQLHGQLYASGAMARLDSCNNCTCVSGKMACTSERCPVACGWSPWTLWSLCSCSCNVGIRRRFRAGTAPPAAFGGAECQGPTMEAEFCSLRPCPGPVPGMCPRDKQWLDCAQGPASCAELSAPRGTNQTCHPGCHCPSGMLLLVSPRGHPGPLGASVQPPVALPGAIGTGSVPGAGGWGPWGPWSHCSRSCGGGLRSRTRACDQPPPQGLGDYCEGPRAQGEVCQALPCPVTNCTAIEGAEYSPCGPPCPRSCDDLVHCVWRCQPGCYCPPGQVLSSNGAICVQPGHCSCLDLLTGQRHHPGARLARPDGCNHCTCLEGRLNCTDLPCPDCGGGQSLHPCGQPCPRSCQDLSPGSVCQPGSVGCQPTCGCPLGQLSQDGLCVPPAHCRCQYQPGAMAPSFVPSTCVAGILQCQEVPDCPDPGVWSSWGPWEDCSVSCGGGEQLRSRRCARPPCPGPARQSRTCSTQVCREAGCPAGRLYRECQPGEGCPFSCAHVTQQVGCFSEGCEEGCHCPEGTFQHRLACVQECPCVLTAWLLQELGATIGDPGQPLGPGDELDSGQTLRTSCGNCSCAHGKLSCSLDDCFEADGGFGPWSPWGPCSRSCGGLGTRTRSRQCVLTMPTLSELPVCPGPGCGAGNCSWTSWAPWEPCSRSCGVGQQRRLRAYRPPGPGGHWCPNILTAYQERRFCNLRACPEAGCPAGMEVVTCANRCPRRCSDLQEGIVCQDDQVCQKGCRCPKGSLEQDGGCVPIGHCDCTDAQGHSWAPGSQHQDACNNCSCQAGQLSCTAQPCPPPTHCAWSHWSAWSPCSHSCGPRGQQSRFRCGPGLASRSGSCPCLMAKADPTCNSTFLHLDTQGCYSGPCPDSCQWSLWGPWSPCQVPCSGGFRLRWREAEALCGGGCREPWAQDRKLQRRALPSTCVNESLVCPHQECPVLGPWSAWSSCSAPCGGGTMERHRTCEGGPGVAPCQAQDTEQRQECNLQPCPECPPGQVLSACATSCPCLCWHLQPGAICVQEPCQPGCGCPGGQHSLPWGLTLTLEEQAQELPPGTVLTRNCTRCVCHGGAFSCSLVDCQGEIVPPGETWQQVAPGELGLCEQTCLEMNATKTQSNCSSARASGCVCQPGHFRSQAGPCVPEDHCECWHLGRPHLVRHRTPSATTHPFLTPSLPICL

(19) United States
(12) Patent Application Publication
(10) Pub. No.: US 2003/0054485 A1
(43) Pub. Date: Mar. 20, 2003

(54) Jelly belly genes and their uses

(76) Inventors: Matthew Peter Scott, Stanford, CA (US); Joseph Benjamin Weiss, Portland, OR (US)

Correspondence Address: BOZICEVIC, FIELD & FRANCIS LLP, 200 MIDDLEFIELD RD, SUITE 200, MENLO PARK, CA, 94025, US

(21) Appl. No.: 10/213,509

(22) Filed: Aug. 6, 2002

Related U.S. Application Data
(60) Provisional application No. 60/311,720, filed on Aug. 9, 2001.

(51) Int. Cl. C12P 21/02; C12N 5/06; C07H 21/04; C12N 9/64; C12N 15/85

(52) U.S. Cl. 435/69.1; 435/320.1; 435/325; 435/455; 435/226; 536/23.2

(57) ABSTRACT

Isolated nucleotide compositions and sequences are provided for jelly belly (jeb) genes. The jeb nucleic acid compositions find use in identifying homologous or related genes; in producing compositions that modulate the expression or function of its encoded protein, Jeb; for gene therapy; mapping functional regions of the protein; and in studying associated physiological pathways. Jeb is required for visceral mesoderm migration and differentiation. Its transcription is directly regulated by Tin. jeb is required for a signal to be transduced from somatic mesoderm to visceral mesoderm. The protein encoded by jeb is secreted from somatic muscle precursors and taken up by visceral muscle precursors. The expression of jeb in neurons later in embryogenesis is indicative that Jeb signaling plays additional roles in the development of the nervous system.

The earliest patterning events of embryonic development have been the objects of intense research, including the mechanisms that establish the anterior-posterior axis as well as the dorsal-ventral axis of the early embryo. Significant understanding of the genes that are required to translate this early positional information into various germ tissues like ectoderm and mesoderm has also been obtained. However, understanding of the developmental events that follow the specification and subdivision of the mesoderm, organogenesis, is notably less complete. Many questions still remain as to how organs form and what genes are required for the complex processes of migration and differentiation that give rise to physiologically functional tissues.

How the actions of genes, for example transcription factors, contribute to the development of diverse mesoderm-derived tissues such as muscles, heart, and blood, is an important and largely unsolved question in cell type determination and organogenesis. It is also a question of clinical significance, as is highlighted by the association of mutations in human NKX2.5, a tin homolog, with a variety of congenital heart lesions. Similarly, TBX5 mutations cause Holt-Oram syndrome and TBX1 mutations cause the cardiac defects in DiGeorge syndrome. Tin, a member of the NK family of homeodomain proteins, is required for organogenesis of the embryonic heart and visceral mesoderm. It is one of a number of transcription factors whose functions in mesoderm development are conserved from insects to mammals. Besides tin and bagpipe (bap), Drosophila homologs of vertebrate Nkx genes, other notable examples include the structurally and functionally related fly and mammalian Mef2 genes, GATA factors, and TBX family genes.

Morphogenesis of the Drosophila mesoderm commences during the first stage of gastrulation. Cells invaginate along the ventral aspect of the embryo. These cells lose their initial epithelial character and spread as mesenchymal cells along the inside of the ectoderm, migrating dorsolaterally away from the ventral midline. Cells that do not migrate to their proper positions, adjacent to the ectoderm, cannot receive the proper inductive signals. Inductive signals from the ectoderm to the mesoderm then refine the patterns of expression of tissue-specific mesoderm transcription factors. The inductive interactions subdivide the early mesoderm into groups of cells that will give rise to the heart, somatic muscle, visceral muscle, fat body, hemocytes, and gonads. Multiple signaling pathways, some used more than once or in combinations, specify the progenitors of diverse mesoderm-derived tissues. Following the early subdivision of the mesoderm into its constituent cell types, mesoderm cells perform coordinated migrations to form organs.

The characterization of genes acting in these early developmental stages is of great interest for research and clinical purposes. In particular, encoded proteins that act as intercellular signals are of particular interest. The number of molecular classes that can act as such signals is small, and the identification of new classes is of great interest.

### SUMMARY OF THE INVENTION

Isolated nucleotide compositions and sequences are provided for jelly belly (jeb) genes. The jeb nucleic acid compositions find use in identifying homologous or related genes; in producing compositions that modulate the expression or function of its encoded protein, Jeb; for gene therapy; mapping functional regions of the protein; and in studying associated physiological pathways. Jeb is required for visceral mesoderm migration and differentiation. Its transcription is directly regulated by Tin. jeb is required for a signal to be transduced from somatic mesoderm to visceral mesoderm. The protein encoded by jeb is secreted from somatic muscle precursors and taken up by visceral muscle precursors. The expression of jeb in neurons later in embryogenesis is indicative that Jeb signaling plays additional roles in the development of the nervous system.

### BRIEF DESCRIPTION OF THE DRAWINGS

FIGS. 1A to 1C. A screen for genes downstream of Tin identifies Tin response elements. (A) Schematic diagram of the strategy for identifying Tin regulated genes. Genetic selection in yeast identifies fragments of genomic DNA that are recognized by the homeodomain of Tin. A hybrid protein that contains the homeodomain of Tin fused to a yeast GAL4 activation domain is expressed in yeast cells. When this protein binds a fragment of Drosophila genomic DNA upstream of the selectable marker, His3, it allows transcription of the selectable marker. (B) The results of a pilot screen that covers 15% of the Drosophila genome. Six fragments were identified, most of them repeatedly, as recognition sites for the Tin-GAL4 hybrid protein. All the fragments contain a core recognition sequence for NK2 class homeodomains. Four of the fragments lie adjacent to known or newly characterized genes. jeb and ind were first identified in this screen. msh is a homeobox gene expressed in dorsal somatic mesoderm, a tissue that develops under Tin control. (C) Reporter constructs containing the fragments identified in the yeast screen drive expression of lacZ in the mesoderm. (1). Dorsal view of a stage 16 embryo showing tin mRNA expression; and (5). Lateral view of tin mRNA expression in stage 11 embryo. At these stages tin expression is restricted to precardiac mesoderm (5) and a subset of heart cells (1). (2). A dorsal view of a stage 16 transgenic embryo transformed with the Fragment A reporter construct. This reporter expresses a nuclearly localized βgalactosidase. lacZ expression is visualized by anti-βgal antibody staining. The reporter is expressed in dorsal somatic mesoderm and the heart, all Tin-dependent tissues. Fragment A maps next to msh which is also expressed in dorsal somatic mesoderm at this stage. (3). A lateral view of a stage 11 transgenic embryo with the Fragment A reporter construct. At this stage expression of the reporter is observed in dorsal pre-cardiac and somatic mesoderm. (4). Ectopic expression of Tin in the pattern of engrailed activates ectopic expression of the reporter construct. A stage 11 transgenic embryo transformed with a reporter construct driven by Fragment A. Ectopic expression of Tin is sufficient to activate the Fragment A reporter construct in the pattern of engrailed. (6). A lateral view of a stage 11 transgenic embryo showing expression of the Fragment B reporter construct. The Fragment B reporter construct is expressed in all of the muscular derivatives of the mesoderm. Fragment B lies adjacent to jeb, which is expressed just in ventral somatic muscle.

FIGS. 2A to 2D. Map of the jeb locus with the transcript structure and predicted protein product. (A) The jeb locus encompasses approximately 30 kb of genomic DNA. In early embryos two transcripts are detected from the locus as shown in (B). The structures of the two early embryonic transcripts are shown in (A). Both contain the same open reading frame. Later in embryogenesis a larger transcript of about 12 kb is detectable. Whole mount in situ hybridization (FIG. 8) implies that this larger transcript is restricted to the central nervous system. (B) RNA blot showing the size and time course of jeb mRNA accumulation. Each lane was loaded with 2 mg of poly-A selected RNA. During the stages of embryogenesis when jeb is first required two size classes of the transcript are detected. The predominant transcript is 3.2 kb, a larger less abundant transcript of 6.6 kb contains the same open reading frame. (C) The predicted protein product of the jeb locus is shown. The first 20 amino acids are hydrophobic and predicted to function as a secretory signal sequence. Close to the carboxy terminus of the protein there is a type A LDL receptor repeat. This portion of the protein is encoded in a single exon. The carboxy terminus of the protein is hydrophobic, but based on its length is not predicted to be membrane spanning. (D) Alignment of the type A LDL receptor repeat of Jelly Belly with two closely related proteins.

FIG. 3. Whole mount in situ hybridization with anti-sense jeb probe shows early expression in the mesoderm. (A) and (C). Lateral views of early stage 9 embryos stage-matched with ventral views of embryos shown in (B) and (D). In all panels anterior is to the left. Jeb expression is first observed in segmentally repeated clusters of ventral mesodermal cells. (C) and (D): Persistence of jeb mRNA expression in stage 10 embryos. Segmental variation in level of expression and the dorsal-ventral position of jeb-expressing cells are also evident. (E) and (F): Ectopic expression of tin is sufficient to ectopically activate expression of jeb. Lateral views of stage 12 embryos hybridized with an antisense probe to jelly belly. In the embryo shown in (F) tin mRNA is ectopically expressed throughout the mesoderm when it would normally be restricted to precursors of the heart. Misexpression of Tin throughout the mesoderm is sufficient to activate ectopic expression of jeb. As shown in (E), jeb mRNA is not normally detectable at this stage of development.

FIG. 4. Jeb is required for the development of midgut muscles. (A) and (B). Lateral views of stage 14 embryos stained with anti-myosin heavy chain antibody. In (A) the midgut musculature of a wild-type embryo is evident and indicated by the arrows. In (B) no midgut musculature is detected with the anti-myosin antibody in a jeb mutant. (C) and (D): Dorsal views of stage 14 embryos stained with an antibody against the myogenic transcription factor Dmef2. In (C) the wild-type embryo shows normal anti-Dmef2 staining of visceral muscles marked by arrows. The jeb mutant shown in (D) demonstrates that Jeb is required for visceral muscle development and that the defect in jeb mutants precedes differentiation. (E) and (F). Endoderm specification and longitudinal migration are normal in jeb mutants but dorsal and ventral endoderm migrations are defective. In (E) the endoderm of a wild-type embryo is stained with an anti-Hindsight antibody. These cells previously migrated centrally from the termini of an early embryo to lie within a tube of visceral muscle cells. In the jeb mutant embryo (F) endoderm precursors have migrated in the anterior-posterior axis to reach the middle of the embryo but they fail to migrate dorsally. Failure of dorsal migration produces a hole in the endoderm whose margin is marked by the arrows. (G) and (H). Increased numbers of somatic muscle precursors are found in jeb mutants. Anti-Dmef2 staining of wild-type (G) and jeb mutant (H) embryos. In jeb mutants more nuclei are observed in the positions of somatic muscle precursors. Later in development the patterning of somatic muscles is normal. (I-K). Fas3, an early marker of visceral mesoderm differentiation is not expressed in jeb mutants. Stage 12 embryos are shown in lateral views stained with an antibody against Fas 3. In a wild-type embryo (l), Fas3 is robustly expressed in visceral mesoderm. In a jeb mutant embryo (J) no Fas3 protein is detectable at this stage. (K). A jeb mutant embryo in which a cDNA transgene has been expressed throughout the mesoderm. Expression of the transgene rescues expression of Fas3 in the visceral mesoderm.

FIG. 5. Visceral mesoderm is normally specified but fails to migrate in jeb mutants. (A) and (B). Comparison of stage 10 wild-type and jeb mutant embryos both stained with anti-Bap antibody to mark early visceral mesoderm. The wild-type embryos in (A), (C), and (E) are also stained with an anti-βgalactosidase antibody in segmentally repeated stripes. The anti-β gal staining allows genotype determination of the embryos; jeb mutant embryos [(B), (D) and (F)] lack detectable βgal protein. Jelly Belly is not required for the specification of visceral mesoderm precursors; Bap expression is normal in the mutants. By mid stage 11 the Bap expressing cells have migrated to form continuous sheets (C); and at higher magnification (E). This contrasts with the jeb mutant embryo (D) and at higher magnification (F). In the absence of Jeb function Bap expressing cells persist as discreet clusters.

FIG. 6. jeb mRNA is expressed in ventral somatic mesoderm adjacent to dorsal, visceral mesoderm precursors. Whole-mount in situ hybridization with anti-sense probes to bap (in red) and jeb (in blue) in wild-type embryos. Jeb function is required in visceral mesoderm cells but the gene is transcribed in somatic mesoderm cells. (A) and (B) are stage 10 embryos, lateral and ventral views. jeb mRNA is expressed in clusters of cells that interdigitate with the bap expressing cells. (C). A lateral view of a stage 11 embryo. At this stage both the jeb and the bap-expressing cells form continuous stripes of cells with the bap-expressing cells lying immediately adjacent and dorsal to the jeb expressing cells.

FIG. 7. Jeb protein is secreted from cells that synthesize it and taken up by cells that require it. (A) and (B). An antibody against Jeb protein demonstrates two patterns of staining in stage 10 embryos. In ventral mesoderm cells both diffuse and punctate cytoplasmic staining is apparent. In dorsal mesoderm, just punctate staining is observed. (C) and (D). Simultaneous staining with anti-Jeb antibody and whole mount in situ hybridization with anti-sense probes for jeb (C) and bap (D) mRNAs. Simultaneous staining for Jeb protein and jeb mRNA (C) demonstrates that diffuse anti-Jeb staining is found in the ventral mesoderm cells that express jeb mRNA and therefore synthesize Jeb protein. Punctate Jeb staining occurs in dorsal mesoderm cells that do not express detectable jeb mRNA. These cells therefore accumulate Jeb protein that was synthesized in the neighboring ventral mesoderm cells. (D). The dorsal mesoderm cells that accumulate Jeb protein are visceral mesoderm precursors that express bap mRNA. The punctate anti-Jeb staining (D) is associated with cells that express the visceral mesoderm marker bap, these are the cells that require Jeb function. (E). Jeb protein is secreted from Drosophila tissue culture cells and found in the culture medium. Jeb protein is detectably more abundantly in the culture medium than in the intracellular fraction shown next to it. 1×10⁶ cells were grown in 2 ml of medium for 72 hours. The medium was removed and the cells lysed in 2 ml of lysis buffer (see Experimental Procedures). For each lane 2.5 μl of medium or lysis buffer was loaded per lane. The difference in mobility and sharpness of resolution in SDS-page implies that Jeb protein is post-translationally modified coincident with secretion. Anti-Tubulin staining of the same fractions is shown as a control for cell lysis and release of intracellular proteins into the culture medium. Minimal Tubulin is found in the culture medium fraction.

FIG. 8. Visceral mesoderm cells do not take up Jeb protein in the P-element induced jeb mutant. (A) and (B). Comparison of wild-type and jeb mutant stage 10 embryos in lateral views, stained with anti-Jeb antisera. In the P-element induced jeb mutant anti-Jeb staining is restricted to ventral mesoderm and is observed in only one, diffuse pattern. (C) and (D). Comparison of the same embryos at higher magnification. In (C) the two patterns of anti-Jeb staining are apparent. In (D) the ventral, punctate anti-Jeb staining associated with bap-expressing cells is absent in the mutant.

FIG. 9. jeb mRNA is expressed in the embryonic CNS and Jeb protein is transported along axons. Panel A shows a ventro-lateral view of a stage 16 embryo hybridized with an anti-sense probe to jeb mRNA that was visualized with a fluorescent chromophore. jeb mRNA is detected in scattered cells throughout the central nervous system. Panels B and C show ventral and lateral views respectively of stage 17 embryos stained with anti-Jeb antibodies. The protein distribution shows Jeb protein associated with axons that form two longitudinal tracts as well as axons that extend laterally into the peripheral nervous system. Panel D shows a lateral view of a stage 17 jeb mutant embryo stained with anti-Jeb antibodies. The protein distribution in jeb mutants does not track axons as in Panel C, but resembles the distribution of the mRNA as in Panel A.

### DESCRIPTION OF THE SPECIFIC EMBODIMENTS

Nucleic acid compositions encoding jelly belly (jeb) are provided. They are used in identifying homologous or related genes; in producing compositions that modulate the expression or function of its encoded protein; for gene therapy; mapping functional regions of the protein; and in studying associated physiological pathways.

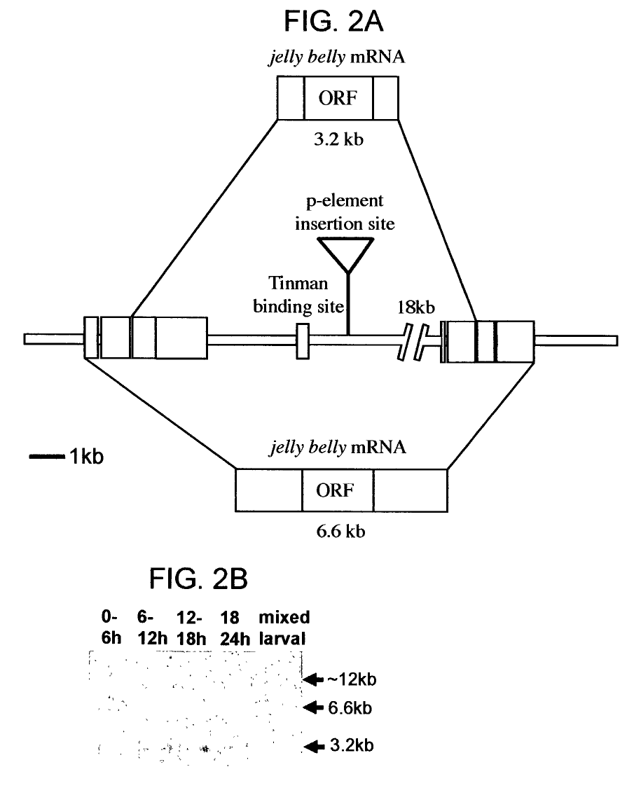

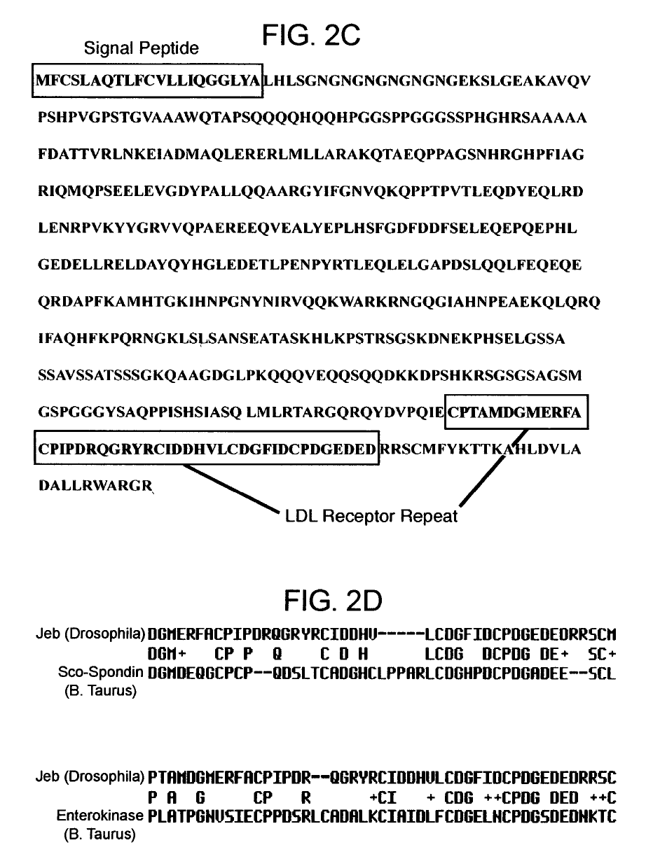

jeb encodes a secreted protein that contains an LDL receptor repeat. The signal sequence and other sequence features is shown in FIG. 2C. The protein also contains an LDL repeat motif, which is implicated in receptor mediated endocytosis, and which is an essential functional domain. One vertebrate homolog of jeb is scospondin, which has been found to be secreted in cow brains, and in some embodiments of the invention the sequence of the bovine sco-spondin will be excluded.

jeb mutants form visceral mesoderm precursors but they fail to migrate or differentiate; and no visceral muscles form. Jeb protein is produced in somatic muscle precursors and taken up by visceral muscle precursors. jeb reveals a signaling process in which somatic muscle precursors support the proper migration and differentiation of visceral muscle cells. Later in embryogenesis jeb is transcribed in neurons, and Jeb protein is found in axons. Expression patterns in vertebrates and flies are similar, where jeb is expressed in the gut, eye and brain, and may be expressed in vascular tissue. The gene is expressed during development, and is required for smooth muscle to migrate, and differentiate. Modulation of expression of jeb finds use in the treatment of hypertension, intimal hyperplasia, for example atherosclerosis, restenosis; pyloric stenosis; etc. jeb is also implicated in interneuronal communication at the synapse, and the integration of sensory information, and finds use in the treatment of neurologic disorders.

The neural function of jeb has a non-developmental, synaptic-modulatory function. It is required for integration of sensory information and associative “learning”. The jeb gene and gene product are therefore useful in modulating such neural function.

### Characterization of Jeb

Homologs of jeb are identified by any of a number of methods. For example, a fragment of the Drosophila cDNA may be used as a hybridization probe against a cDNA library from the target organism of interest, where low stringency conditions are used. Alternatively, searches may be performed against genetic databases using any convenient algorithm for alignment. The probe may be a large fragment, or one or more short degenerate primers. Such sequences are selected from regions that are not likely to diverge over evolutionary time and are of low degeneracy. The complementary binding sequence will usually be at least 14 nucleotides, preferably at least about 17 nucleotides and usually at least about 30 nucleotides. A probe of particular interest is the LDL receptor repeat, which corresponds to residues 494-537 of the Jeb amino acid sequence; residues 1700-1832 of the nucleotide sequence (SEQ ID NO:1).

Between distantly related species, for example between the invertebrate Drosophila and a mamalian species, the level of sequence identity may be fairly low, and protein sequence comparisons may be preferred, so as to minimize the effects of codon redundancy. For example, the sequence identity for amino acids in the LDL receptor repeat is from about 40% to 50% between the LDL receptor repeat region of SEQ ID NO:1, and representative mammalian counterparts.

Between more closely related species, for example between two mammalian sequences, such as mouse and human; cow and mouse; etc., the level of sequence identity is higher, for example being at least about 80% over a length of 100 of more residues in the nucleotide sequence. Representative sequences from the mouse and human jeb genes are provided in SEQ ID NO:3; and SEQ ID NO:4, respectively; where nt sequences encoding the protein of SEQ ID NO:05 are also of interest.

Nucleic acids having sequence similarity to the provided jeb genetic sequences can also be detected by hybridization under low stringency conditions, for example, at 50° C. and 6×SSC (0.9 M NaCl/0.09 M Na citrate) and remain bound when subjected to washing at 55° C. in 1×SSC (0.15 M NaCl/0.015 M Na citrate). Substantial sequence identity may be determined by hybridization under stringent conditions, for example, at 50° C. or higher and 0.1×SSC (15 mM NaCl/01.5 mM Na citrate). Nucleic acids having a region of substantial identity to the provided jeb sequences, e.g. allelic variants, genetically altered versions of the gene, etc., bind to the provided jeb sequences under stringent hybridization conditions. By using probes, particularly labeled probes of DNA sequences, one can isolate homologous or related genes. The source of homologous genes may be any species, e.g. primate species, particularly human; rodents, such as rats and mice, canines, felines, bovines, ovines, equines, yeast, nematodes, etc.

Conveniently, amplification reactions are used to generate an initial probe, which can then be used to hybridize to a library; for rapid amplification of cloned ends (RACE); etc. One or more of the resulting clones may then be used to rescreen the library to obtain an extended sequence, up to and including the entire coding region, as well as the non-coding 5′- and 3′-sequences. As appropriate, one may sequence all or a portion of the resulting cDNA coding sequence. The source of mRNA for a cDNA library will use cells where jeb is known to be expressed, for example mesodermal tissue, such as smooth muscle, eye, neurons, gut, etc.

Between species in a group, e.g. different mammals, homologs have substantial sequence similarity, i.e. at least 75% sequence identity between nucleotide sequences, in some cases 80 or 90% sequence identity, and may be as high as 95% sequence identity between closely related species. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10. In general, variants of the invention have a sequence identity greater than at least about 65%, preferably at least about 75%, more preferably at least about 85%, and may be greater than at least about 90% or more as determined by the Smith-Waterman homology search algorithm as implemented in MPSRCH program (Oxford Molecular). Exemplary search parameters for use with the MPSRCH program in order to identify sequences of a desired sequence identity are as follows: gap open penalty: 12; and gap extension penalty: 1.

### JEB NUCLEIC ACID COMPOSITIONS

Nucleic acids encoding jeb may be cDNA or genomic DNA or a fragment thereof. The term “jeb gene” shall be intended to mean the open reading frame encoding specific jeb polypeptides, introns, as well as adjacent 5′ and 3′ non-coding nucleotide sequences involved in the regulation of expression, up to about 20 kb beyond the coding region, but possibly further in either direction. The gene may be introduced into an appropriate vector for extrachromosomal maintenance or for integration into the host.

The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, removed by nuclear RNA splicing, to create a continuous open reading frame encoding a Jeb protein.

A genomic sequence of interest comprises the nucleic acid present between the initiation codon and the stop codon, as defined in the listed sequences, including all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 3′ and 5′ end of the transcribed region. The genomic DNA may be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression.

The sequence of the 5′ flanking region may be utilized for promoter elements, including enhancer binding sites, that provide for developmental regulation in tissues where jeb is expressed. The tissue specific expression is useful for determining the pattern of expression, and for providing promoters that mimic the native pattern of expression. Naturally occurring polymorphisms in the promoter region are useful for determining natural variations in expression, particularly those that may be associated with disease.

Alternatively, mutations may be introduced into the promoter region to determine the effect of altering expression in experimentally defined systems: Methods for the identification of specific DNA motifs involved in the binding of transcriptional factors are known in the art, e.g. sequence similarity to known binding motifs, gel retardation studies, etc. For examples, see Blackwell et al. (1995) Mol Med 1: 194-205; Mortlock et al. (1996) Genome Res. 6: 327-33; and Joulin and Richard-Foy (1995) Eur J Biochem 232:620-626.

The regulatory sequences may be used to identify cis acting sequences required for transcriptional or translational regulation of jeb expression, especially in different tissues or stages of development, and to identify cis acting sequences and trans acting factors that regulate or mediate jeb expression. Such transcription or translational control regions may be operably linked to a jeb gene in order to promote expression of wild type or altered jeb or other proteins of interest in cultured cells, or in embryonic, fetal or adult tissues, and for gene therapy.

The nucleic acid compositions of the subject invention may encode all or a part of the subject polypeptides. Double or single stranded fragments may be obtained of the DNA sequence by chemically synthesizing oligonucleotides in accordance with conventional methods, by restriction enzyme digestion, by PCR amplification, etc. For the most part, DNA fragments will be of at least 15 nt, usually at least 18 nt, more usually at least about 50 nt. Such small DNA fragments are useful as primers for PCR, hybridization screening probes, etc. Larger DNA fragments, i.e. greater than 100 or 250 nt are useful for production of the encoded polypeptide. For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

The jeb genes are isolated and obtained in substantial purity, generally as other than an intact, naturally occurring chromosome. Usually, the DNA will be obtained substantially free of other nucleic acid sequences that do not include a jeb sequence or fragment thereof, generally being at least about 50%, usually at least about 90% pure and are typically “recombinant”, i.e. flanked by one or more nucleotides with which it is not normally associated on a naturally occurring chromosome.

The DNA may also be used to identify expression of the gene in a biological specimen. The manner in which one probes cells for the presence of particular nucleotide sequences, as genomic DNA or RNA, is well established in the literature and does not require elaboration here. DNA or mRNA is isolated from a cell sample. The mRNA may be amplified by RT-PCR, using reverse transcriptase to form a complementary DNA strand followed by polymerase chain reaction amplification using primers specific for the subject DNA sequences. Alternatively, the mRNA sample is separated by gel electrophoresis, transferred to a suitable support, e.g. nitrocellulose, nylon, etc., and then probed with a fragment of the subject DNA as a probe. Other techniques, such as oligonucleotide ligation assays, in situ hybridizations, and hybridization to DNA probes arrayed on a solid chip may also find use. Detection of mRNA hybridizing to the subject sequence is indicative of jeb gene expression in the sample.

The sequence of a jeb gene, including flanking promoter regions and coding regions, may be mutated in various ways known in the art to generate targeted changes in promoter strength, sequence of the encoded protein, etc. The DNA sequence or protein product of such a mutation will usually be substantially similar to the sequences provided herein, i.e. will differ by at least one nucleotide or amino acid, respectively, and may differ by at least two but not more than about ten nucleotides or amino acids. The sequence changes may be substitutions, insertions or deletions. Deletions may further include larger changes, such as deletions of a domain or exon. Other modifications of interest include epitope tagging, e.g. with the FLAG system, HA, etc. For studies of subcellular localization, fusion proteins with green fluorescent proteins (GFP) may be used.

Techniques for in vitro mutagenesis of cloned genes are known. Examples of protocols for site specific mutagenesis may be found in Gustin et al., Biotechniques 14:22 (1993); Barany, Gene 37:111-23 (1985); Colicelli et al., Mol Gen Genet 199:537-9 (1985); and Prentki et al., Gene 29:303-13 (1984). Methods for site specific mutagenesis can be found in Sambrook et al., Molecular Cloning: A Laboratory Manual, CSH Press 1989, pp. 15.3-15.108; Weiner et al., Gene 126:35-41 (1993); Sayers et al., Biotechniques 13:592-6 (1992); Jones and Winistorfer, Biotechniques 12:528-30 (1992); Barton et al., Nucleic Acids Res 18:7349-55 (1990); Marotti and Tomich, Gene Anal Tech 6:67-70 (1989); and Zhu, Anal Biochem 177:120-4 (1989). Such mutated genes may be used to study structure-function relationships of jeb, or to alter properties of the protein that affect its function or regulation.

### Jeb Polypeptides

The subject gene may be employed for producing all or portions of Jeb polypeptides. For expression, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to a jeb gene, or may be derived from exogenous sources.

The peptide may be expressed in prokaryotes or eukaryotes in accordance with conventional ways, depending upon the purpose for expression. For large scale production of the protein, a unicellular organism, such as E. coli B. subtilis, S. cerevisiae, insect cells in combination with baculovirus vectors, or cells of a higher organism such as vertebrates, particularly mammals, e.g. COS 7 cells, may be used as the expression host cells. In some situations, it is desirable to express the jeb gene in eukaryotic cells, where the Jeb protein will benefit from native folding and post-translational modifications. Small peptides can also be synthesized in the laboratory. Peptides that are subsets of the complete jeb sequence, e.g. peptides of at least about 8 amino acids in length, usually at least about 12 amino acids in length, and may be as many as about 20 amino acids in length, up to substantially the length of the intact protein, may be used to identify and investigate parts of the protein important for function, or to raise antibodies directed against these regions. The expressed polypeptide may comprise the complete unprocessed protein, or the processed mature protein, lacking the signal sequence. Other domains of interest include the LDL receptor repeat.

With the availability of the protein or fragments thereof in large amounts, by employing an expression host, the protein may be isolated and purified in accordance with conventional ways. A lysate may be prepared of the expression host and the lysate purified using HPLC, exclusion chromatography, gel electrophoresis, affinity chromatography, or other purification technique. The purified protein will generally be at least about 80% pure, preferably at least about 90% pure, and may be up to and including 100% pure. Pure is intended to mean free of other proteins, as well as cellular debris.

The expressed Jeb polypeptides are used for the production of antibodies, where short fragments provide for antibodies specific for the particular polypeptide, and larger fragments or the entire protein allow for the production of antibodies over the surface of the polypeptide. Antibodies may be raised to the wild-type or variant forms of Jeb. Antibodies may be raised to isolated peptides corresponding to specific domains, e.g. the LDL receptor repeat; to denatured or to native protein, etc.

Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, e.g. in conjunction with in vitro affinity maturation.

### Diagnostic Uses

The subject nucleic acid and/or polypeptide compositions may be used to analyze a genetic sample for the expression of jeb, or for the presence of polymorphisms in jeb, which may be associated with a disease state or genetic predisposition to a disease state. Biochemical studies may be performed to determine whether a sequence polymorphism in a jeb coding region or control regions is associated with disease. Disease associated polymorphisms may include mutations that alter expression level, that affect protein function, etc.

Changes in the promoter or enhancer sequence that may affect expression levels of jeb can be compared to expression levels of the normal allele by various methods known in the art. Methods for determining promoter or enhancer strength include quantitation of the expressed natural protein; insertion of the variant control element into a vector with a reporter gene such as β-galactosidase, luciferase, chloramphenicol acetyltransferase, etc. that provides for convenient quantitation; and the like.

A number of methods are available for analyzing nucleic acids for the presence of a specific sequence. mRNA or genomic DNA may be used directly, or may be amplified. The nucleic acids may be amplified by conventional techniques, such as the polymerase chain reaction (PCR), to provide sufficient amounts for analysis. The use of the polymerase chain reaction is described in Saiki, et al. (1985) Science 239:487, and a review of techniques may be found in Sambrook, et al. Molecular Cloning: A Laboratory Manual, CSH Press 1989, pp. 14.2□14.33. Alternatively, various methods are known in the art that utilize oligonucleotide ligation as a means of detecting polymorphisms, for examples see Riley et al. (1990) N.A.R. 18:2887-2890; and Delahunty et al. (1996) Am. J. Hum. Genet. 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid is analyzed by one of a number of methods known in the art. The nucleic acid may be sequenced by dideoxy or other methods, and the sequence of bases compared to a wild-type jeb sequence. Hybridization with the variant sequence may also be used to determine its presence, by Southern blots, dot blots, etc. The hybridization pattern of a control and variant sequence to an array of oligonucleotide probes immobilized on a solid support, e.g. commercially available microarrays, may also be used as a means of detecting the presence of variant sequences. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Alternatively, where a polymorphism creates or destroys a recognition site for a restriction endonuclease, the sample is digested with that endonuclease, and the products size fractionated to determine whether the fragment was digested. Fractionation is performed by gel or capillary electrophoresis, particularly acrylamide or agarose gels.

Screening for mutations in jeb may be based on the functional or antigenic characteristics of the protein. Protein truncation assays are useful in detecting deletions that may affect the biological activity of the protein. Various immunoassays designed to detect polymorphisms in Jeb proteins may be used in screening. Where many diverse genetic mutations lead to a particular disease phenotype, functional protein assays have proven to be effective screening tools. The activity of the encoded Jeb protein may be determined by comparison with the wild-type protein.

Antibodies specific for a Jeb polypeptide may be used in staining or in immunoassays. Samples, as used herein, include biological fluids such as cerebrospinal fluid, tears, saliva, blood, dialysis fluid and the like; organ or tissue culture derived fluids; and fluids extracted from physiological tissues. Also included in the term are derivatives and fractions of such fluids. The cells may be dissociated, in the case of solid tissues, or tissue sections may be analyzed. Alternatively a lysate of the cells, e.g. smooth muscle cells, may be prepared.

Diagnosis may be performed by a number of methods to determine the absence or presence or altered amounts of normal or abnormal Jeb in patient cells. For example, detection may utilize staining of cells or histological sections, performed in accordance with conventional methods. Cells are permeabilized to stain cytoplasmic molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g. fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc.

### Modulation of Gene Expression

The jeb genes, gene fragments, or the encoded protein or protein fragments are useful in gene therapy to treat disorders associated with jeb defects. Expression vectors may be used to introduce the jeb gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

As Jeb is a secreted protein, therapeutic uses may also include administration of the protein systemically, or to the extracellular microenvironment in a localized manner. To decrease Jeb activity, blocking agents may be administered extracellularly, e.g. antibodies that bind to Jeb, and the like.

The gene or Jeb protein may be introduced into tissues or host cells by any number of routes, including injection, viral infection, microinjection, or fusion of vesicles. Jet injection may also be used for intramuscular administration, as described by Furth et al. (1992) Anal Biochem 205:365-368. The DNA may be coated onto gold microparticles, and delivered intradermally by a particle bombardment device, or “gene gun” as described in the literature (see, for example, Tang et al (1992) Nature 356:152-154), where gold microprojectiles are coated with the Jeb protein or DNA, then bombarded into skin cells.

Antisense molecules can be used to down-regulate expression of jeb in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences. Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases, e.g. morpholino derivatives have found wide use in the inhibition of gene expression.

In addition to antisense, small interfering RNA (siRNA) duplexes can be used to inhibit expression of jeb genes. siRNA are double stranded RNA molecules of at least about 18 nucleotides, and may be up to the length of the complete mRNA. Preferred siRNA for use in mammalian cells are from about 18 to 30 nucleotides, preferably from about 21 to 22 nucleotides in length. For example, see Elbashir et al. (2001) Nature 411:494-498.

### Genetically Altered Cell or Animal Models for Jeb Function

The subject nucleic acids can be used to generate transgenic animals or site specific gene modifications in cell lines. Transgenic animals may be made through homologous recombination, where the normal jeb locus is altered. Alternatively, a nucleic acid construct is randomly integrated into the genome. Vectors for stable integration include plasmids, retroviruses and other animal viruses, YACs, and the like.

The modified cells or animals are useful in the study of jeb function and regulation. For example, a series of small deletions and/or substitutions may be made in the jeb gene to determine the role of different exons in smooth muscle development, synaptic signaling, receptor mediated endocytosis, etc. Of interest are the use of jeb to construct transgenic animal models for neural and muscle development, where expression of jeb is specifically reduced or absent, e.g. in muscle cells, neurons, etc. Specific constructs of interest include anti-sense jeb, which will block jeb expression and expression of dominant negative jeb mutations. A detectable marker, such as lac Z may be introduced into the jeb locus, where upregulation of jeb expression will result in an easily detected change in phenotype.

One may also provide for expression of the jeb gene or variants thereof in cells or tissues where it is not normally expressed or at abnormal times of development. In addition, by providing expression of Jeb protein in cells in which it is not normally produced, one can induce changes in cell behavior.

DNA constructs for homologous recombination will comprise at least a portion of the jeb gene with the desired genetic modification, and will include regions of homology to the target locus. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keyed et al. (1990) Methods in Enzymology 185:527-537.

For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture. The transgenic animals may be any non-human mammal, such as laboratory animals, domestic animals, etc. The transgenic animals may be used in functional studies, drug screening, etc., e.g. to determine the effect of a candidate drug on smooth muscle development, neural signaling, developmental abnormalities, etc.

### In Vitro Models for Jeb Function

Drug screening may be performed using an in vitro model, a genetically altered cell or animal, or purified Jeb protein. One can identify ligands or substrates that bind to, modulate or mimic the action of Jeb. Areas of investigation include the development of treatments for vascular diseases involving smooth muscle cells, restenosis, atherosclerosis, etc., investigation into the development of smooth muscle; modulation of neural signaling, etc.

Drug screening identifies agents that provide a replacement for Jeb function in abnormal cells. Agents that mimic its function are predicted to inhibit the process of oncogenesis. Conversely, agents that reverse Jeb function may stimulate controlled growth and healing. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions.

The term “agent” as used herein describes any molecule, e.g. protein or pharmaceutical, with the capability of altering or mimicking the physiological function of Jeb. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Other assays of interest detect agents that mimic jeb function. The level of jeb activity is determined by a functional assay, for example by binding to cells previously shown to have a receptor for Jeb.

The compounds having the desired pharmacological activity may be administered in a physiologically acceptable carrier to a host for treatment of smooth muscle disorders, e.g. hyperproliferative disorders of smooth muscle cells such as neointimal hyperplasia, which may include restenosis, atherosclerosis, etc., developmental abnormalities attributable to a defect in jeb function, etc. The compounds may also be used to enhance jeb function. The inhibitory agents may be administered in a variety of ways, orally, topically, parenterally e.g. subcutaneously, intraperitoneally, by viral infection, intravascularly, etc. Topical treatments are of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

### Experimental

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

Results

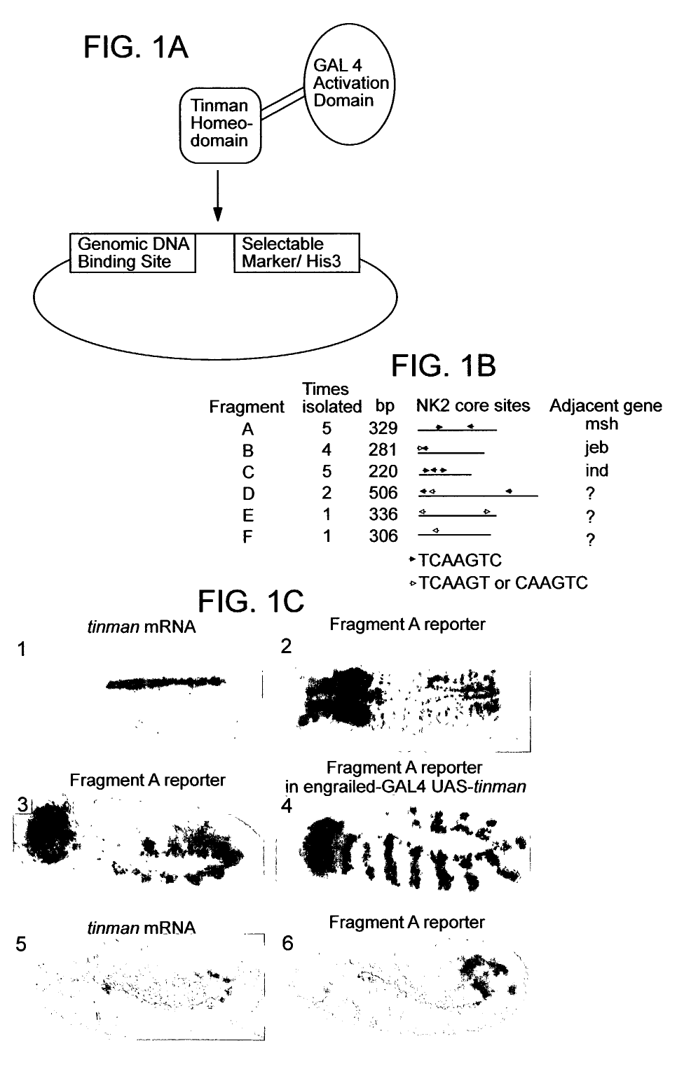

A screen for genes downstream of Tin. A screen was performed to identify genes that are transcriptionally regulated by the homeodomain protein Tin. The method, shown schematically in FIG. 1A, relies on genetic selection in yeast for a protein-DNA interaction. A library was screened that represents 15% of the Drosophila genomic DNA and six DNA fragments were obtained that satisfied genetic criteria in yeast for Tin binding sites (FIG. 1B). Most of the genomic DNA fragments were isolated multiple times. Sequence analysis confirmed the presence of core recognition sites for NK class homeodomains in all of the fragments (FIG. 1B). To show that these fragments represent Tin responsive enhancers in vivo, the encoded products were tested for the ability to drive expression of a reporter gene in patterns consistent with Tin regulation.

Based on the reporter construct results and the genes that are located adjacent to the Tinman binding sites, the screen was found to be specific for genes regulated by Tinman or closely related genes. Four fragments identified in the yeast screen (A-D in FIG. 1B) were inserted upstream of a lacZ reporter. Three of the four reporter constructs are expressed as transgenes in patterns consistent with Tin regulation (FIG. 1C). The Fragment A reporter transgene is expressed in dorsal somatic and cardiac mesoderm (FIG. 1C2). This fragment maps near msh, a gene that is also expressed in dorsal somatic mesoderm. It was confirmed that the fragment A reporter construct is responsive to Tinman by crossing it into embryos in which Tinman was misexpressed in the striped pattern of engrailed. Misexpression of Tin is sufficient to activate expression of the reporter construct (FIG. 1, C3 vs. 4). A second reporter construct, containing Fragment B, is expressed throughout the mesoderm similar to the earliest expression of Tin. This fragment lies adjacent to jelly belly (jeb), a gene expressed in the mesoderm in a more restricted pattern than the reporter construct. The expression pattern, structure, and function of jeb are described below. Fragment C, the one of the four that did not support expression of the reporter, lies adjacent to ind. ind was first identified in this screen and is a target of negative regulation by Vnd/NK2, a protein structurally similar to Tin.

Identification of the jelly belly gene and the structure of the jeb locus. The Tin-binding site that led to jeb (Fragment B in FIG. 1B) contains two adjacent Tin/NK2 class homeodomain recognition sites oriented as an imperfect inverted repeat. This genomic fragment was mapped to interval 48E1 of the D. melanogaster polytene chromosome 2R using in situ hybridization. Sequence alignment of the same fragment with the genomic sequence of Drosophila confirmed the localization. The Tin binding sites lie adjacent to a P element insertion within a large intron of the jeb gene, and the P element is responsible for a lethal mutation (FIG. 2A).

Multiple cDNAs were isolated by hybridization to the jeb genomic DNA. Sequence analysis of the cDNAs, and developmental RNA blots (FIG. 2B) demonstrate two size classes of transcripts derived from the jeb locus during early to mid embryogenesis. Later in embryogenesis a third, larger, transcript is detected. The jeb function in the embryonic visceral mesoderm precedes the appearance of the larger transcript. The early embryonic transcripts contain the same open reading frame as the large transcript, differing only in 5′ and 3′ untranslated regions. The predicted protein product of the jeb locus contains a secretory signal sequence and a single LDL receptor repeat motif (FIGS. 2C and D). In the region of the LDL receptor repeat, Jeb is most similar to two bovine proteins called Scospondin and enterokinase (FIG. 2D). The functions of the bovine proteins are unknown.

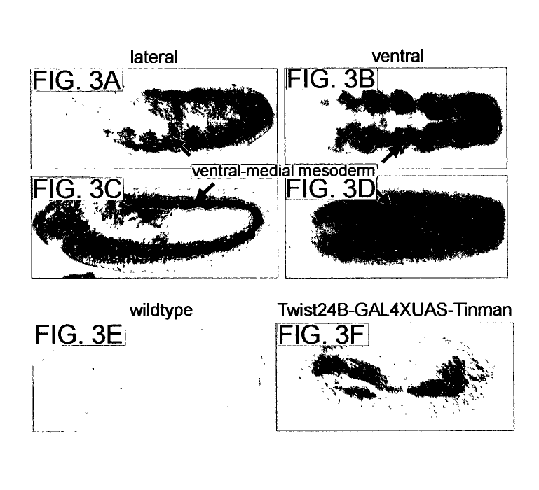

jeb is expressed in ventral mesoderm. The pattern of jeb transcription was determined using whole mount in situ hybridization to embryos. jeb mRNA is first detectable at stage 8 in repeated segmental.clusters of ventral mesoderm cells (FIG. 3). These cells are precursors of somatic muscle. Subsequently jeb is transcribed in two roughly parallel, continuous bands in the ventral mesoderm. By stage 12 jeb mRNA is no longer detectable in the mesoderm.

Tin is sufficient but not necessary for jeb expression in the mesoderm. jeb was identified as a putative Tin target gene. jeb expression in tin mutant embryos is scarcely different from wild type, though it may be somewhat reduced. Tin activation of jeb transcription is likely to be redundant with some other regulator of mesoderm development. To test the sufficiency of Tin for activating jeb, embryos in which tin was ectopically expressed were assessed for ectopic jeb expression. Misexpression of tin in the ectoderm with an engrailed GAL4 driver did not alter jeb expression. Misexpression of tin throughout the mesoderm is sufficient to activate jeb expression at a late time (stage 12) when it is not expressed in wild-type embryos (FIG. 3F), and in tissues where jeb is normally not expressed. That Tin can activate jeb in the mesoderm but not ectoderm suggests that a cofactor present in the mesoderm is required for Tin-mediated activation of jeb transcription. The expression domains of tin and jeb imply that Tin's role in the regulation of jeb is restricted to the earliest stages of jeb expression, since at late stage 10 Tin is found only in dorsal mesoderm and Jeb is found in ventral mesoderm.

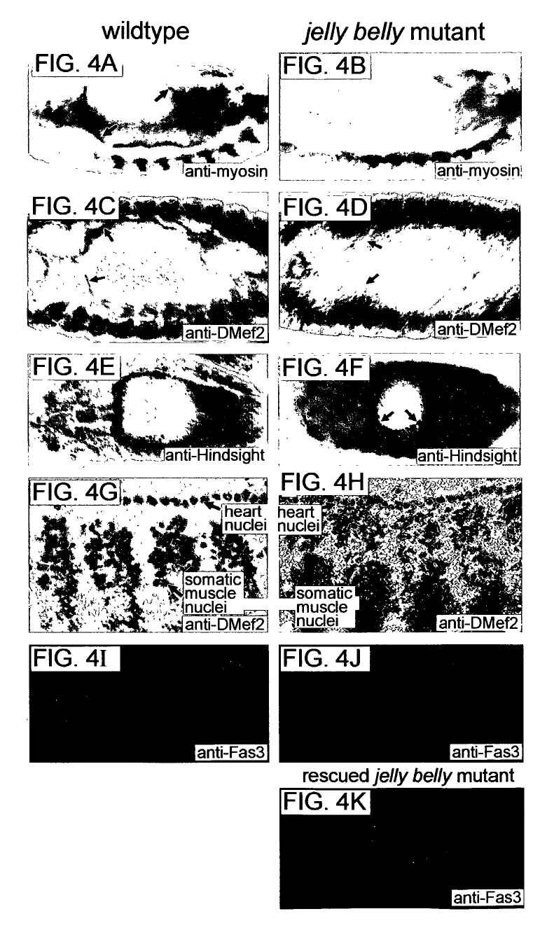

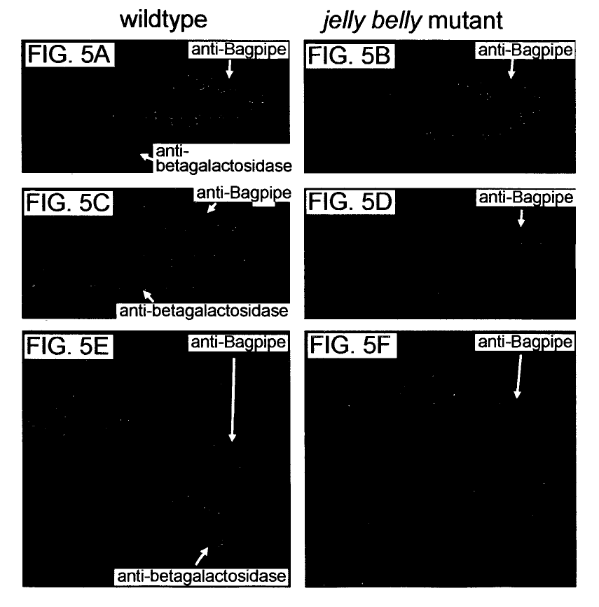

jeb is required for visceral mesoderm migration and differentiation. The P element transposon insertion that interrupts the jeb transcription unit (FIG. 2A) is the cause of a recessive lethal jeb mutation that affects mesoderm development. Precise excision of the P insert allows survival and reverts the phenotype. The phenotype is due to loss of jeb function because: 1) we detect no other transcripts from the genomic region that contains both jeb and the P element insertion, and 2) the phenotype of the P element allele is observed when and where Jeb is produced, and 3) we can rescue the mutant phenotype by driving expression of a jeb cDNA transgene in mutant embryos (FIG. 4I-K). These observations indicate that the phenotype associated with the P element insertion is solely attributable to loss of Jeb function.

The function of jeb in mesoderm development was assessed in homozygous jeb mutant embryos. jeb function is required for visceral mesoderm development (FIG. 4). Anti-myosin heavy chain antibody staining shows the thin layer of mesoderm overlying the yolk in the gut of wild-type embryos (FIG. 4A, arrows). In jeb mutants no differentiated visceral mesoderm is detectable (FIG. 4B). Other terminally differentiated muscular components of the mesoderm, the somatic muscles and dorsal vessel or heart, are not affected in jeb mutants. Similarly other mesoderm-derived tissues, the fat body and hemocytes, appear to develop normally in jeb mutants. The morphology of the fat body is disrupted, most likely as a secondary consequence of abnormal midgut morphogenesis.

Myosin heavy chain is a marker of differentiated muscle, so the jeb mutation could affect differentiation or a prior step in visceral mesoderm development. To evaluate the visceral mesoderm phenotype at earlier stages, jeb mutant embryos were stained with an antibody against D-Mef2, an evolutionarily conserved myogenic transcription factor that is required for muscle differentiation. D-Mef2 is expressed quite early in all muscle lineages of the Drosophila embryo, and its early mesoderm expression is normal in jeb mutants. In jeb mutants no D-Mef2 staining is detectable where the visceral mesoderm should be (FIG. 4C vs. D). The visceral mesoderm defect in jeb mutants must occur prior to differentiation, and also prior to midgut morphogenesis. D-Mef2 staining of jeb mutants reveals an apparent increase in somatic muscle precursors, consistent with the hypothesis that visceral mesoderm precursors assume a somatic mesoderm fate in jeb mutants (FIG. 4G vs. H).

Endoderm development in jeb mutants is not primarily or severely affected. Antibodies against Hindsight protein, a marker of the midgut endoderm epithelium (FIG. 4E), were used to determine whether the lack of visceral mesoderm in jeb mutants reflects the complete absence of midgut. Despite the absence of visceral mesoderm in jeb mutants, the endoderm is specified normally and migrates longitudinally to form two continuous, longitudinal bands (FIG. 4F). Subsequent dorsal and ventral endoderm migration does not occur normally in jeb mutants. Dorsal and ventral endoderm migration is dependent on the visceral mesoderm, so the defect may well be a secondary consequence of the visceral mesoderm defect.

Visceral mesoderm precursors are specified in the dorsal, lateral mesoderm, prior to midgut morphogenesis, under the combined positive inductive influences of Dpp and Hh produced by the overlying ectoderm. Specification of visceral mesoderm precursors also requires Bap, a homeodomain protein related to Tin. If jeb was required to generate visceral mesoderm cells, this would be apparent as a change in bap expression. However Bap protein accumulates normally in jeb mutants during the period of visceral mesoderm specification (FIG. 5A vs. B). Visceral mesoderm precursors that are correctly positioned and contain Bap protein are normally specified in jeb mutants.

In wild-type embryos bap-expressing cells, initially specified as segmentally repeated, discrete clusters, commence midgut morphogenesis by migrating longitudinally to form two parallel continuous bands. In jeb mutants bap-expressing cells fail to migrate to form these two continuous bands. Instead the cells persist as discrete clusters (FIG. 5, C vs. D and E vs. F). Shortly after the longitudinal migration of the bap-expressing cells, Bap protein decays away and Fas3, an early marker of visceral mesoderm, is produced. Fas3, a structural protein, is at this stage tissue-specific and as such is a useful early marker of differentiation. In jeb mutants Fas3 is weakly and transiently produced. At the germ band retraction stage, when Fas3 production is robust in wild-type embryos, it is absent in jeb mutants (FIG. 4I vs. J).

Visceral mesoderm cell fate in jeb mutants. jeb is transcribed in somatic mesoderm cells, yet Bap staining shows that visceral mesoderm precursors form normally but fail to migrate in the absence of jeb function. There is no evidence of visceral mesoderm in jeb mutants after stage 11, so what becomes of the bap-expressing cells? They could undergo programmed cell death. Transcription patterns of three genes that serve as markers of apoptosis, grim, hid, and reaper (Chen et al., 1996; Grether et al., 1995; White et al., 1994), are the same in jeb mutants as in wild-type embryos. TUNEL staining was done to confirm the result, and again no evidence of increased programmed cell death was found.

It has been proposed that visceral mesoderm cells in bap mutant embryos assume a somatic mesoderm fate. D-Mef2 stains of jeb mutant embryos show increased numbers of nuclei in positions consistent with an increase in somatic muscle precursors (FIG. 4G vs. H). Anti-myosin staining of jeb mutants shows that no major disruption of somatic muscle patterning occurs in jeb mutants. It seems most likely that, in the absence of jeb function, visceral mesoderm cells default to a somatic mesoderm fate. The extra somatic muscle precursors may become incorporated into the normal somatic muscle pattern.

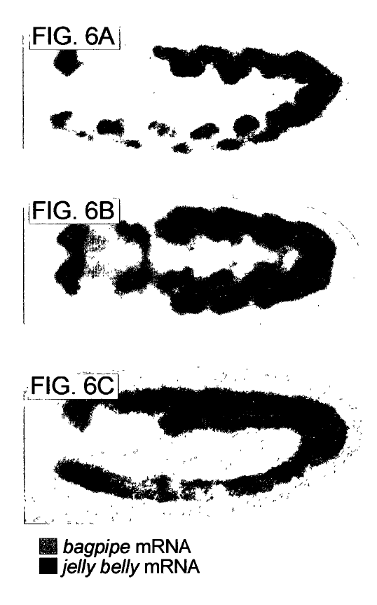

Evidence that Jeb functions non-cell autonomously. jeb is required for visceral mesoderm development, but not for somatic muscle, fat body, or hemocyte development. To understand how Jeb might function biochemically we determined where, within the mesoderm, jeb is expressed in relation to early visceral mesoderm. jeb is clearly expressed in ventral and medial mesoderm immediately adjacent to the visceral mesoderm cells that require Jeb function (FIG. 6A-C). The cells that express jeb are thought to be somatic muscle precursors. jeb mRNA is initially produced in repeated clusters of cells ventral to clusters of bap-expressing cells (FIG. 6A). At stage 10 jeb-expressing cells surround the visceral mesoderm and fill in the gaps between the clusters of bap expression. By mid-stage 11, jeb and bap expression are in juxtaposed layers of mesodermal tissue (FIG. 6C).

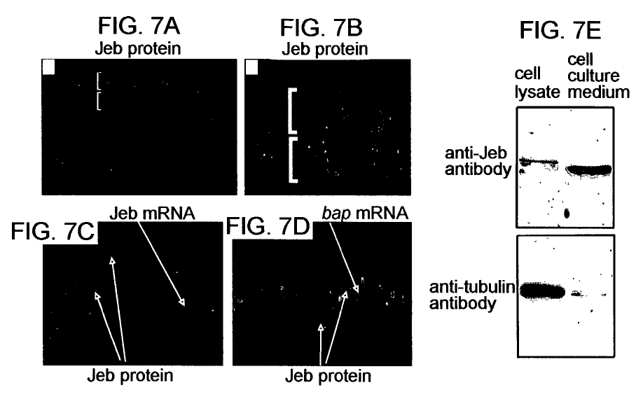

Jeb protein is secreted and taken up by visceral mesoderm cells. Jeb protein contains a signal sequence and a LDL receptor repeat. Both of these structural motifs imply that Jeb protein is secreted from somatic mesoderm precursor cells and acts in the extracellular compartment or upon other cells. To test this hypothesis we generated polyclonal antisera against Jeb protein made in bacteria. The antisera were affinity purified and found to specifically recognize a protein found in the mesoderm where and when jeb mRNA is transcribed (FIG. 7A, C). If Jeb protein is a signal from somatic to visceral mesoderm precursors, bap expressing cells that are dependent on jeb function but do not transcribe jeb could contain Jeb protein. Double-label staining for Jeb protein and bap RNA clearly show Jeb protein in bap-transcribing cells that do not synthesize jeb mRNA (FIG. 7D, arrows).

Jeb protein is secreted from tissue culture cells (FIG. 7E). Extracts of Drosophila tissue culture cells producing Jeb were compared to protein found in their medium. The bulk of the Jeb protein was found outside the cells. The secreted protein migrates as a broad band. Thus Jeb protein is clearly detectable, evidently in a post-translationally modified form, in the culture medium.

A truncated Jeb protein is not taken up by visceral mesoderm precursors. The P element that is integrated into the jeb locus interrupts the transcription unit in a large intron (FIG. 2A). Transcription of jeb upstream of the integration site would generate a mRNA predicted to encode a protein of about 50 kD. In mutant flies, affinity-purified sera detect a truncated Jeb protein with an apparent molecular weight of 45 kD. The predicted mutant protein would contain the secretory signal sequence but would lack the type A LDL receptor repeat domain. We investigated whether the truncated protein moves into visceral mesoderm cells like the wild-type protein.

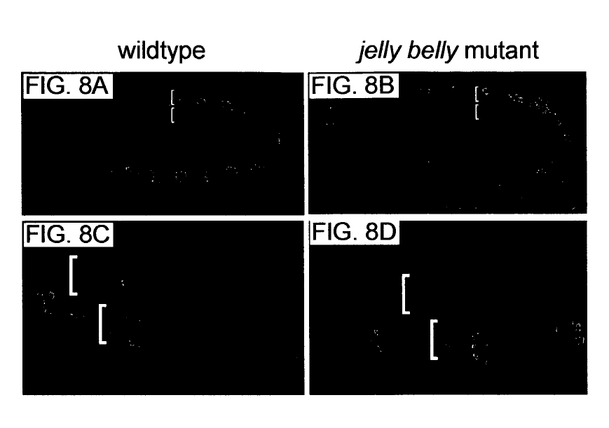

Antibody stains of jeb mutant embryos reveal two notable differences with respect to wild-type protein distribution. First, the truncated protein accumulates to lower levels than the wild-type protein. Second, visceral mesoderm precursors do not take up the truncated protein (FIG. 8C vs. D). The only detectable protein in mutant embryos is in or adjacent to the cells that make it. We conclude that the type A LDL receptor repeat is necessary for either secretion and stabilization of Jeb, or is required for recognition of the protein by a specific receptor for Jeb.

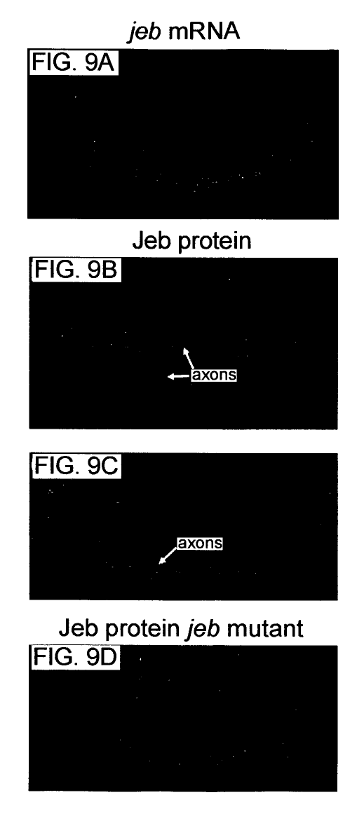

A role for Jeb signaling in CNS development or function. Developmental signals are often employed at multiple times and in multiple tissues. Examples include the roles of Hh and Wg signals in early embryonic segment polarity and later in imaginal disc patterning. jeb functions as a novel signal and like other signals is active in more than one time and place. The first site of expression outside the mesoderm is the embryonic CNS (FIG. 9). At stage 16, jeb mRNA is detected in a subset of embryonic neurons that are distributed throughout the ventral nerve cord. Jeb protein, detected with affinity-purified antisera, appears in a small set of longitudinal axons of the CNS as well as some lateral axons that exit to the PNS. These data show that Jeb signaling in the CNS and PNS may be used for communication among a restricted group of neurons.

In the P-element induced mutation of jeb the protein distribution is strikingly different from wild type (FIG. 9C vs. D). In the jeb mutants the protein distribution resembles the pattern of MRNA expression (FIG. 9A). By extrapolation from the mesoderm results, the altered protein distribution in jeb mutants implies that the axonal staining observed in wild-type embryos represents transport of the protein in neurons that have taken up the protein, as opposed to Jeb secreting cells. This signal transport resembles that observed for Hh in the developing eye (Huang and Kunes, 1996).

jeb uncovers a new signaling system Mutants that lack jeb form visceral mesoderm precursors normally but these cells do not migrate longitudinally, instead they persist as discrete clusters. The precursors also fail to produce a tissue-specific marker of visceral mesoderm, and they do not go on to form any detectable visceral musculature. The protein product of the jeb gene is contains a secretory signal sequence and a type A LDL receptor repeat, and serves as an extracellular signaling molecule. jeb is not transcribed in visceral mesoderm precursors, the cells affected by jeb mutations, but is transcribed in adjacent ventral mesoderm cells, which are somatic muscle precursors. Jeb protein, in contrast, is detectable both in the cells that synthesize it and in the cells that respond to it. The observed distribution of Jeb protein supports a signaling mechanism for Jeb function in development. In tissue culture Jeb protein is secreted from cells that synthesize it, and the protein is readily detected in the culture medium. Jeb protein is specifically taken up by visceral mesoderm cells, as opposed to other equally proximate tissues, implying that uptake. depends on a tissue-specific receptor.

The above data demonstrate that jeb is a component of a new signaling system, which may be a positive migratory or differentiation signal for visceral mesoderm precursors. Jeb could be a ligand for a tissue-specific receptor, or could collaborate with one or more other proteins to stimulate production of a positive migratory or differentiation signaling ligand. The uptake of Jeb protein by visceral mesoderm suggests that Jeb itself is the ligand or part of the ligand.

Regulation of jeb transcription. jeb was identified in a screen for genes that are downstream targets of transcriptional regulation by the NK homeodomain protein Tin. tin mutants have no detectable heart or visceral mesoderm, and have severely disrupted dorsal somatic muscles. Vertebrate homologs of tin appear to play related roles in the development of vertebrate mesoderm. jeb responds to Tin transcriptional activation in the mesoderm but Tin is not necessary for jeb expression. Loss-of-function mutations in Tin are not associated with significantly perturbed expression of jeb by whole mount in situ hybridization. The sufficiency of Tin misexpression to activate jeb transcription implies that Tin plays an early and redundant function in the regulation of jeb. Other regulators that may play roles in the regulation of jeb include the bHLH protein Twist and the Pax domain protein Pox Meso.

Jeb and other LDL repeat-containing proteins. The presence of a single LDL receptor repeat in Jeb suggests functional similarity between Jeb and other LDL receptor repeat-containing proteins. One other well-characterized LDL receptor repeat containing protein may be functionally related to Jeb: the product of the C. elegans gene Mig-13 which, like Jeb, contains a single LDL receptor repeat. Structurally Mig-13 differs from Jeb in that it contains both a CUB and a transmembrane domain not found in Jeb. Mig-13 function, however, resembles Jeb in two notable ways. First, Mig-13 is required non-cell autonomously, like Jeb. Second, Mig-13 is necessary for anterior migration of developing neurons in C. elegans, a positive migratory function that resembles the effects of Jeb. Mig-13 is produced locally along the anterior-posterior body axis under the control of specific Hox genes, and appears to guide migrations in a concentration-dependent manner.

Signaling systems are often used repeatedly in a variety of developmental contexts. Jeb is no exception to this rule. jeb is expressed in the ventral nerve cord during embryogenesis and late in larval development in the developing visual system and central nervous system. Jeb is therefore probably also used as a signal later in development.

### Experimental Procedures

Yeast screen for genomic DNA bound by Tinman, and gene discovery. The screen for Tinman binding sites in Drosophila genomic DNA was performed as described in Weiss et al. (1998) Genes Dev 12, 3591-602.

Molecular Biology. Molecular biologic techniques including genomic and cDNA library screening, DNA sequencing and RNA blotting were all according to standard protocols as in Sambrook et al., (1989). Molecular Cloning: A Laboratory Manual., Second Edition: Cold Spring Harbor Press).

In situ hybridization. Whole-mount in situ hybridization to Drosophila embryos was performed according to the protocol of Lehmann and Tautz, 1994. Proteinase K digestion was omitted from all in situ hybridizations. The procedure for two-color in situ hybridizations was according to Weiss et al. (1998) supra.

Antibody production. A bacterial GST-fusion protein containing amino acids 41 through 355 of Jeb was constructed in the expression vector pGEX-4T-1 (Pharmacia). This construct was transformed into BL21 cells (Stratagene). The cells were grown at 26° C., lysed in MTPBS (150 mM NaCl; 4 mM NaH₂PO₄; 16 mM Na₂HPO₄) with 0.2 mg/ml of lysozyme and protease inhibitors (Protease inhibitor cocktail, Roche). Recombinant protein was partially purified by chromatography on glutathione-sepharose 4B (Amersham Pharmacia). Partially purified recombinant protein was further purified by preparative SDS-PAGE. Electrophoretically purified recombinant protein was employed as an immunogen to inoculate rabbits on a standard schedule (Josman Laboratories). 161 days after the initial innoculation the rabbit was sacrificed and serum collected. Raw serum was affinity-purified by chromatography on an AminoLink (Pierce) agarose column to which purified, bacterially expressed GST-fusion protein had been attached.

Immunohistochemistry. Antibody staining with anti-βgal, anti-myosin, anti-DMef2, anti-Hindsight, anti-Fas3, anti-Bap and anti-Jeb was performed according to standard protocols Patel, et al. (1994) lmagining Neuronal Subsets and Other Cell Types in Whole-Mount Drosophila Embryos and Larvae Using Antibody Probes. In Drosophila melanogaster Practical Uses in Cell and Molecular Biology, L. S. B. Goldstein and E. A. Fyrberg, eds. (San Diego: Academic Press), pp. pp. 445-487. The anti-βgal antibody was used at a titer of 1:1000 (Cappel) anti-rabbit HRP-conjugated secondary antibody at 1:400 (Jackson), and staining visualized by diaminobenzadine (DAB) reaction in FIG. 1, or Cy5 Renaissance Tyramide Signal amplification (NEN) in FIG. 5. The mouse anti-myosin monoclonal antibody (kindly provided by D. Kiehart) was used at a titer of 1:5, anti-mouse biotin-conjugated secondary antibody at 1:400 (Jackson), and signal amplification by incubation with the ABC Reagent (Vectastain) and DAB reaction. The anti-DMef2 antibody (kindly provided by B. Patterson) was used at a titer of 1:1000, anti-rabbit biotin-conjugated secondary at 1:400 (Jackson) and signal amplification by incubation with ABC reagent (Vectastain) and DAB reaction. The mouse monoclonal anti-Hindsight antibody (kindly provided by H. Lipshitz) was used at a titer of 1:10, anti-mouse biotin-conjugated secondary at 1:400 (Jackson) and signal amplification by incubation with ABC reagent (Vectastain) and DAB reaction. Mouse monoclonal anti-Fas3 antibody (kindly provided by the Developmental Studies Hybridoma Bank at the University of Iowa) was used at a titer of 1:5, biotin-conjugated anti-mouse secondary at 1:400 (Jackson) signal amplification by incubation with ABC reagent (Vectastain) and Cy5 Renaissance Tyramide Signal Amplification (NEN). Anti-Bap staining with rabbit anti-serum (kindly provided by M. Frasch) was performed at a titer of 1:100, biotin-conjugated anti-rabbit secondary at 1:400 (Jackson) and signal amplification by incubation with ABC reagent (Vectastain) and Cy5 Renaissance Tyramide Signal Amplification (NEN). Anti-Jeb anti-serum was used at a titer of 1:1000, and staining visualized with TRITC-conjugated anti-rabbit antibody at 1:400.

Double fluorescent in situ hybridization and antibody labelling. The protocol for fluorscent in situ hybridization and antibody labeling is contained in Knirr et al. (1999) Development 126, 4525-35. In situ hybridizations to mRNA were visualized with Cy3 Renaissance Tyramide Signal Amplification (NEN) in FIG. 8 or Cy5 Renaissance Tyramide Signal Amplification (NEN) in FIG. 9. Antibody stains were visualized with TRITC conjugated secondary antibodies (Jackson).

Tissue culture secretion assay and protein blot. S2 Drosophila tissue culture cells were grown in Schneider's Medium (Gibco) with 10% heat-inactivated fetal calf serum and gentamycin. S2 cells were found to express Jeb protein. For the secretion assay approximately 10⁶ cells were plated in 35 mm-tissue culture plates. The cells were grown in 2 ml of medium for 72 hours. The medium was then removed and any suspended cells removed from the medium by centrifugation. The tissue culture cells remaining on the plate were lysed in 2 ml of insect lysis buffer (10 mM Tris, pH 7.5; 130 mM NaCl; 1% VN Triton X-100; 10 mM NaF; 10 mM Na₂H₂PO₄; 10 mM Na₄P₂O₇) with protease inhibitors (Protease inhibitor cocktail, Roche) on ice for 45 minutes. The cell lysate was cleared by centrifugation at 40,000×g for 45 minutes. Immunoblotting was performed according to standard protocols.

Drosophila methods. The Drosophila line harboring a P element insertion into the jeb locus was obtained from the Bloomington Drosophila Stock Center. Excision of the P element to revert the mutation was performed according to standard protocols. Reporter constructs were created by insertion of genomic DNA fragments into C4pLZ (Wharton and Crews, (1993) Mech Dev 40, 141-54). These constructs were used to derive transgenic Drosophila lines by standard methods. Ectopic expression of Tinman was performed using the GAL4-UAS system (Brand and Perrimon (1993) Development 118, 401-15). The tinman cDNA was inserted into pUAST and tranformed into embryos. Transgenic lines were isolated and crossed to engrailed-GAL4 or twist-24B-GAL4 drivers obtained from the Bloomington Drosophila Stock Center. Rescue of the jeb mutation was performed using the GAL4-UAS system as well. The jeb cDNA was inserted into pUAST and transformed into embryos to generate transgenic Drosophila lines. Rescue of jeb mutants was accomplished by expression of the UAS-jeb cDNA with the twist-24B-GAL4 driver in a jeb mutant background.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in. protein. structure without affecting the biological action. in kind or amount. All such modifications are intended to be included within the scope of the appended claims.

1. An isolated nucleic acid molecule other than a naturally occurring chromosome comprising a sequence encoding a Jeb protein, wherein said nucleic acid is other than bovine.

2. An isolated nucleic acid molecule according to claim 1, wherein said Jeb protein comprises the sequence set forth in SEQ ID NO:2.

3. An isolated nucleic acid molecule according to claim 2, wherein said nucleic acid comprises the nucleotide sequence set forth in any one of SEQ ID NO:1, SEQ ID NO:3; or a fragment thereof.

4. An isolated nucleic acid comprising at least 18 contiguous nucleotides of the sequence of any one of SEQ ID NO:1, SEQ ID NO:3.

5. An isolated nucleic acid comprising at least 50 contiguous nucleotides of the sequence of any one of SEQ ID NO:1,SEQ ID NO:3.

6. An isolated nucleic acid that hybridizes under stringent conditions to the nucleic acid sequence of any one of SEQ ID NO:1, SEQ ID NO:3.

7. An expression cassette comprising a transcriptional initiation region functional in an expression host, a nucleic acid having a sequence of the isolated nucleic acid according to claim 1 under the transcriptional regulation of said transcriptional initiation region, and a transcriptional termination region functional in said expression host.

8. A cell comprising an expression cassette according to claim 7 as part of an extrachromosomal element or integrated into the genome of a host cell as a result of introduction of said expression cassette into said host cell, and the cellular progeny of said host cell.

9. A cell comprising a nucleic acid according to claim 1 as part of an extrachromosomal element or integrated into the genome of a host cell as a result of introduction of said expression cassette into said host cell, and the cellular progeny of said host cell.

10. A method for producing jeb protein, said method comprising:
growing a cell according to claim 8, whereby said jeb protein is expressed; and
isolating said jeb protein free of other proteins.

11. A purified polypeptide composition comprising at least 50 weight % of the protein present as a Jeb protein.

12. A purified polypeptide according to claim 11, wherein said polypeptide comprises the amino acid sequence set forth in SEQ ID NO:2.

13. A purified polypeptide fragment of at least 12 amino acids, and comprising a sequence within SEQ ID NO:2.

14. An antibody specific for the polypeptide of claim 11.

15. A method of. screening for biologically active agents that modulate Jeb function, the method comprising:
combining a candidate biologically active agent with any one of:
(a) a Jeb polypeptide;
(b) a cell comprising a nucleic acid encoding a Jeb polypeptide; or
(c) a non-human transgenic animal model for Jeb gene function comprising one of: (i) a knockout of an Jeb gene; (ii) an exogenous and stably transmitted Jeb gene sequence; or (iii) an Jeb promoter sequence operably linked to a reporter gene; and determining the effect of said agent on Jeb function.
